(19) United States
(12) Patent Application Publication
(10) Pub. No.: US 2023/0151056 A1
(43) Pub. Date: May 18, 2023

(54) EXOSOME PURIFICATION METHOD AND INTEGRATED DEVICE THEREOF

(71) Applicant: Ningbo Yongheng Yaoyao Intelligent Technology Co., Ltd., Ningbo (CN)

(72) Inventor: Jiaming HE, Ningbo (CN)

(21) Appl. No.: 17/659,696

(22) Filed: Apr. 19, 2022

(30) Foreign Application Priority Data
Nov. 18, 2021 (CN) 202111371330.8

(51) Int. Cl. C07K 1/34; C07K 1/16; C07K 1/36; B01D 61/14

(52) U.S. Cl. CPC C07K 1/34; C07K 1/16; C07K 1/36; B01D 61/142; B01D 2311/06

(57) ABSTRACT

A method exosome purification and characterization is contemplated, comprising the steps of secondary two-stage tangential ultrafiltration to produce an extracted solution, pretreatment of the extracted solution for characterization, characterization of the extracted solution to detect particle size and concentration, and freeze-drying of the extracted solution. An exosome purification integrated device is also contemplated. Through the disclosed methods and devices, exosomes may be better purified and characterized in a manner that results in high practical value to overcome the problems associated with conventional exosome purification processes on the market today, including tedious purification processes, long durations, and high costs.

### CROSS-REFERENCE TO RELATED APPLICATIONS

This patent application claims the benefit and priority of Chinese Patent Application No. 202111371330.8, filed on Nov. 18, 2021, the disclosure of which is incorporated by reference herein in its entirety as part of the present application.

### TECHNICAL FIELD

The present disclosure relates to the technical field of biology, and in particular relates to an exosome purification method and an integrated device thereof.

### BACKGROUND

In 2013, the Nobel Prize in Physiology or Medicine was awarded to three scientists for their research in the field of cellular vesicle transport regulation mechanism, after which exosome research gradually became a scientific hotspot. Exosomes are small vesicles of about 30-150 nm in diameter secreted by a living cell, with a typical lipid bilayer structure, and are widely discovered in cell culture supernatants, blood, saliva, urine, semen, amniotic fluid, and other biological fluids. Exosomes carries a variety of proteins, lipids, RNA and other important information, play an important role in cell-to-cell material and information transfer, can regulate cell metabolism, apoptosis and immune regulation, and have utility in the fields of medical cosmetology, regenerative medicine, disease diagnosis, and other industries. Many have hailed exosomes as hailed as the next frontier of cell therapy.

At present, the methods of separation, enrichment and extraction of exosomes are mainly performed as manual operations, with the main technical methods including centrifugation, precipitation, ultrafiltration, immunoaffinity, microfluidic and the like, with quantitative analysis being conducted on the particle size parameters of the exosomes and the like by means of flow cytometry and nanoparticle tracking analysis technology, thus guaranteeing the quality of the exosomes. With the development of science and technology, and especially in applications of exosome-based treatments for enhancing health and beauty, the market demand for exosomes has increased, and the traditional methods of exosome separation technology are insufficient to meet this capacity due to limitations preventing large-scale extraction separation and purification, resulting in short supply of the exosome.

As the separation and extraction of exosome at present are mostly performed as manual operations, the whole separation and extraction process is tedious and takes a long time. Existing automatic separation and extraction solutions are low in output, slow, high in cost, and thus it is difficult to meet the market demand Exosome vendors urgently need a preparation platform capable of obtaining high-purity exosomes quickly, in large quantities, and with high quality. However, conventional ultracentrifugation methods for the exosome purification process are slow, time-consuming, laborious, strongly depend on the experience of operators, poor in repeatability of collection results, and low in recovery rate, all of which limit the industrial production and clinical application of the exosomes produced via such exosome purification processes. For example, in one certain industrial purification process, although quantification, concentration detection and the like can be conducted to achieve a target level of purification, the chromatographic methods used in this industrialized process, such as affinity chromatography and ion exchange chromatography, have certain limitation due to the requirements of a chromatographic column, requiring control of parameters such as PH, buffer and conductivity of a fed sample and the isoelectric point of the purified substance, and the like. Alternative methods such as immunomagnetic bead methods and microfluidic methods are relatively high in cost, poor in economic efficiency, require expensive technical machinery and highly trained operators, and are only suitable for trace purification operations, resulting in difficulties in large-scale preparation. In addition, determination of the content and concentration of exosomes in a raw material sample, and characterization of the particle size and quality of the produced exosomes, have also become urgent requirements of vendors. Independent operation and use of TFF (tangential flow filtration), FPLC (fast protein liquid chromatography) and NTA (nanoparticle tracking analysis) technologies on the market also limit the development of the whole exosome industry, and thus the increasing market demand for exosome-containing products is difficult to meet. Therefore, an integrated device capable of achieving scale production of exosomes and performing quantitative detection and other nanoparticle tracking analysis functions has become an urgent requirements of various scientific research institutions, colleges and universities and enterprise manufacturers. Likewise, developing an integrated device incorporating TFF, FPLC and NTA technologies and having a compatible operation system is also desirable.

### BRIEF SUMMARY

The technical problem to be solved by the present disclosure is to provide an integrated device for exosome purification to solve the problems of low production efficiency and complex operation of the common exosome purification and extraction solutions currently on the market.

To solve the problem, the exosome purification integrated device comprises a tangential flow ultrafiltration system, a fast protein liquid chromatography system, a nanoparticle tracking analysis system and a peristaltic pump for transferring liquid through the system, with the tangential flow ultrafiltration system comprising a first-stage tangential flow ultrafiltration device and a second-stage tangential flow ultrafiltration device which are in communication with each other; with an ultrafiltration membrane arranged in each of the first-stage tangential flow ultrafiltration device and the second-stage tangential flow ultrafiltration device, the a pore size of the ultrafiltration membrane of the first-stage tangential flow ultrafiltration device being greater than a pore size of the ultrafiltration membrane of the second-stage tangential flow ultrafiltration device, and with the second-stage tangential flow ultrafiltration device being connected to an inlet of the fast protein liquid chromatography system, and an outlet of the fast protein liquid chromatography system being connected to the nanoparticle tracking analysis system.

According to one exemplary embodiment of the present disclosure, the first-stage tangential flow ultrafiltration device may be provided with a first-stage inlet and a first-stage outlet; the second-stage tangential flow ultrafiltration device is provided with a second-stage inlet and a second-stage outlet, and the first-stage outlet is communicated with the second-stage inlet.

According to the exemplary embodiment, the nanoparticle tracking analysis system is further communicated with the first-stage inlet.

According to the exemplary embodiment, the fast protein liquid chromatography system is further connected to a freeze-drying refrigeration system.

According to the exemplary embodiment, the freeze-drying refrigeration system comprises a mechanical transfer device, a freeze dryer, and a low-temperature storage space; and the mechanical transfer device is used for transferring a product frozen by the freeze dryer to the low-temperature storage space.

Through the secondary two-stage tangential flow ultrafiltration, the device provided by the present disclosure greatly improves the separation purity of the exosome, has the advantage of shortening the analysis period, improving the separation and purification capacity and increasing the sensitivity; and by adopting a “TFF+FPLC+NTA” “sandwich hamburger” structure model, with all systems being serially configured in series, so that an extraction solution sample passes through TFF, FPLC and NTA under the external pressure of the peristaltic pump to conduct automatic pretreatment, concentration content detection, enrichment purification and particle size analysis operations on the exosomes, and after the purity and quality reach a collection requirement, the enriched and purified liquid is freeze-dried into the freeze-dried powder by a vacuum freeze dryer, with the purity of the exosome in the prepared freeze-dried powder being high.

Another technical problem to be solved by the present disclosure is to provide an exosome purification method to solve the problems of the tedious purification processes, long durations and high costs of the conventional exosome purification method currently on the market.

To solve above problems, an exosome purification method is provided, which comprises the following steps:

S1. secondary two-stage tangential ultrafiltration: introducing an extraction solution containing at least one exosome targeted for extraction into a first-stage tangential flow ultrafiltration device having a first-stage ultrafiltration membrane to obtain from the permeate a first-stage liquid permeate, delivering the first-stage liquid permeate into a second-stage tangential flow ultrafiltration device having a second-stage ultrafiltration membrane to obtain from the retentate an enriched liquid solution;

Wherein the pore size of the first-stage ultrafiltration membrane of the first-stage tangential flow ultrafiltration device is greater than the pore size of the second-stage ultrafiltration membrane of the second-stage tangential flow ultrafiltration device, and wherein the pore size of the second-stage ultrafiltration membrane of the second-stage tangential flow ultrafiltration device is less than the diameter of the at least one exosome targeted for extraction such thus the exosome targeted for extraction can pass through the first-stage ultrafiltration membrane and cannot pass through the second-stage ultrafiltration membrane;

S2. solution pretreatment: introducing the enriched liquid solution into a fast protein liquid chromatograph to obtain an eluent solution;

S3. exosome particle size and concentration detection: mixing the eluent solution obtained in step S2 with a phosphate buffered solution to obtain a mixed solution, and injecting the mixed solution into a nanoparticle tracking analyzer to determine particle size and concentration data associated with the enriched liquid solution;

S4. freeze-drying: freeze drying the enriched liquid solution obtained in step S1 to obtain a freeze-dried powder permeate.

Through the secondary two-stage tangential flow ultrafiltration, the separation purity of the exosome is greatly improved, the method has the advantages of shortening the analysis period, improving the separation and purification capacity and increasing the sensitivity; through the filtration of the first-stage tangential flow ultrafiltration membrane, cells, cell debris and apoptotic bodies in a sample are firstly filtered to obtain the first-stage liquid permeate containing the exosomes, then through the filtration of the second-stage tangential flow ultrafiltration membrane, the first-stage liquid permeate containing the exosomes is subjected to the secondary tangential flow ultrafiltration to obtain from the retentate an enriched liquid containing the exosomes. The nanoparticle tracking analyzer is then used to analyze the enriched liquid and determine the particle size of the exosome in the enriched liquid and, if necessary, to such data to determine whether the enriched liquid must be fed back to the tangential flow ultrafiltration system for re-purification, thus guaranteeing the purification quality. The enriched liquid is freeze-dried to obtain exosome freeze-dried powder with a high purity and recovery rate. High-throughput, large-scale, factory-scale extraction, separation and enrichment of the exosome can be achieved through the method provided by the present disclosure, and thus the problem of insufficient exosome production capacity is solved, meeting the market demand for such a solution. According to the method, by applying a fast protein liquid chromatography (FPLC) technology and a nanoparticle tracking analysis (NTA) technology, and combining with those a mature tangential flow ultrafiltration (TFF) technology, with the tangential flow ultrafiltration method being improved to utilize secondary two-stage tangential ultrafiltration, the purification efficiency is improved beyond the original basis of the fast protein liquid chromatography method. The NTA is integrated to conduct particle size tracking analysis on the enriched exosome. The three technologies are combined to serve as a core step of exosome purification, and thereafter, the extracted exosome is subjected to a freeze-drying operation to finally prepare the exosomes with higher purity. Compared with ultracentrifugation, size exclusion chromatography and immunoprecipitation methods, the ultrafiltration technology described herein has the advantages of being short in time, simple and efficient, with damage to exosomes being more effectively reduced through tangential flow ultrafiltration, and such ultrafiltration technology may be seen to be suitable for separation and purification production of high-purity and high-throughput large-scale industrial samples.

As a preferred solution, wherein in the step S2, the pore size of the first-stage TFF ultrafiltration membrane is greater than or equal to 0.18 μm, and the pore size of the second-stage TFF ultrafiltration membrane is less than or equal to 0.03 μm.

As a preferred solution, specific operations in the step S4 are as follows: adding an excipient into the enriched liquid, bottling the enriched liquid, and then transferring the bottled enriched liquid into a vacuum freeze dryer, and freeze-drying to obtain the freeze-dried powder.

According to a preferred embodiment, in step S2, conditions for chromatography treatment are as follows: the fast protein liquid chromatograph is configured to chromatographically treat the enriched liquid solution using a chromatographic column employing an affinity column, the fast protein liquid chromatograph utilizing an equilibrium liquid comprising 0.06M Tris-HCl and 0.5 M NaCl having a PH of 7.9, an eluant comprising 0.06 M Tris-HCl, 0.5 M NaCl, and 0.6 M imidazole having a PH of 7.9, and a flow rate for equilibrating four column beds and eluting one column bed of 4 mL/min.

According to a preferred embodiment, in step S3, the volume ratio of the eluent solution to the phosphate buffered solution is 6:4.

According to a preferred embodiment, in step S1, the extraction solution containing the targeted exosome comprises one or more of: cell supernatant, blood, urine, saliva, amniotic fluid, urine, and seminal fluid.

According to a preferred embodiment, step S3 further comprises a step of comparing the determined particle size and concentration data to an acceptable particle size and concentration range, and if it is detecting that the determined particle size and concentration data is not within the acceptable particle size and concentration range, performing further tangential ultrafiltration treatment upon the enriched liquid solution, wherein the acceptable particle size and concentration range comprises an concentration greater than or equal to 109/ml, and a mean particle size range of between 30 nm to 150 nm.

### BRIEF DESCRIPTION OF THE DRAWINGS

FIG. 1 is a structure diagram of an exemplary exosome purification integrated device according to the present disclosure;

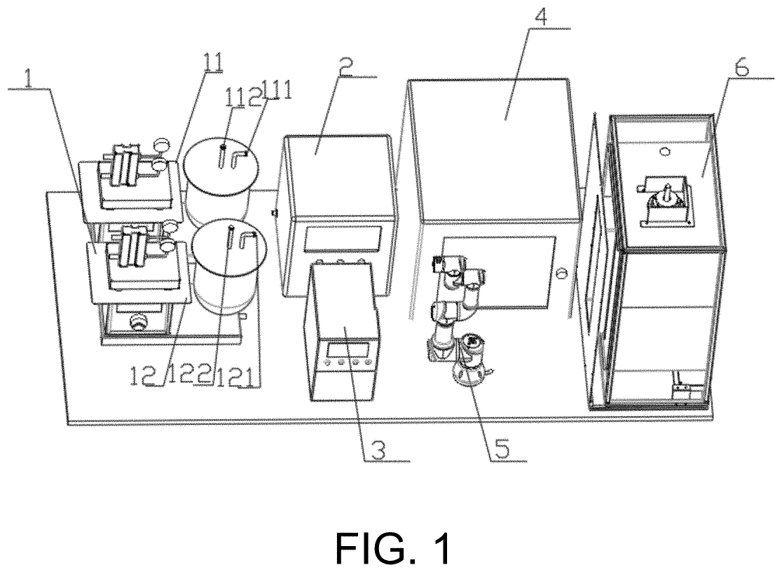

FIG. 2 is a diagram of a working principle of an exemplary embodiment of the method of the present disclosure; and

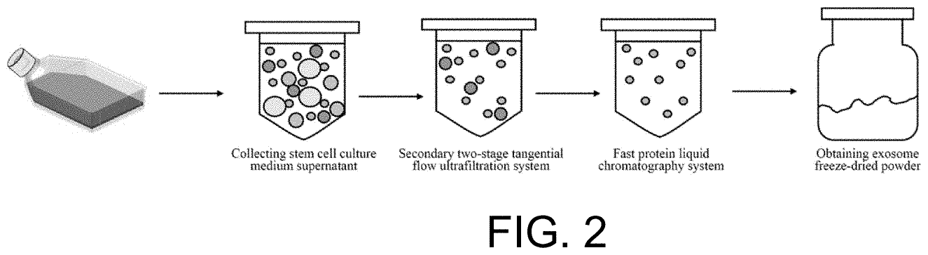

FIG. 3 is a diagram of a working process of an exemplary embodiment of the method of the present disclosure.

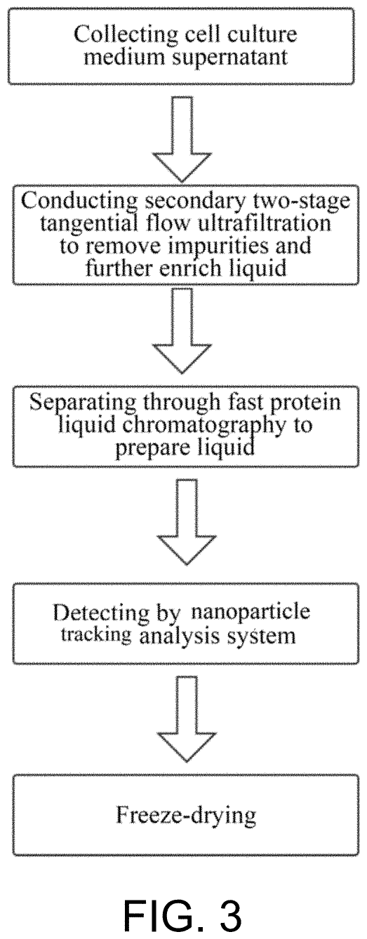

Common reference numerals are used throughout the drawings and detailed description to indicate the same elements, and are as follows:

1—tangential flow ultrafiltration system; 11—first-stage tangential flow ultrafiltration device; 111—first-stage inlet; 112—first-stage outlet; 12—second-stage tangential flow ultrafiltration device; 121—second-stage inlet; 122—second-stage outlet; 2—fast protein liquid chromatography system; 3—nanoparticle tracking analysis system; 4—freeze dryer; 5—mechanical transfer device; 6—low-temperature storage space.

### DETAILED DESCRIPTION

To assist in rendering the objectives, features and advantages of the present disclosure more clear and understandable, the following specific embodiments of the present disclosure are described in detail below with reference to the accompanying drawings.

### Embodiment 1

The embodiment provides an exosome purification integrated device, which specifically comprises a tangential flow ultrafiltration system 1, a fast protein liquid chromatography system 2, a nanoparticle tracking analysis system 3 and a peristaltic pump for transferring liquid through the system, which may be communicated through a pipeline; the peristaltic pump is used for transferring a flow of liquid through the device; by adding extraction solution containing the exosome into the device, the extraction solution sequentially flows through the tangential flow ultrafiltration system 1, the fast protein liquid chromatography system 2 and the nanoparticle tracking analysis system 3 to complete the purification of the exosome.

The tangential flow ultrafiltration system 1 comprises a first-stage tangential flow ultrafiltration device 11 and a second-stage tangential flow ultrafiltration device 12 which are in communication with each other; the first-stage tangential flow ultrafiltration device 11 is provided with a first-stage inlet 111 and a first-stage outlet 112; the second-stage tangential flow ultrafiltration device 12 is provided with a second-stage inlet 121 and a second-stage outlet 122, and the first-stage outlet 112 is communicated with the second-stage inlet 121, thus the introduced extraction solution firstly enters the first-stage tangential flow ultrafiltration device 11 for ultrafiltration, and then flows into the second-stage tangential flow ultrafiltration device 12 for repeated ultrafiltration;

An ultrafiltration membrane is arranged in each of the first-stage tangential flow ultrafiltration device 11 and the second-stage tangential flow ultrafiltration device 12, and the pore size of the ultrafiltration membrane in the first-stage tangential flow ultrafiltration device 11 is greater than the pore size of the ultrafiltration membrane in the second-stage tangential flow ultrafiltration device 12. The pore size of the ultrafiltration membrane in the second-stage tangential flow ultrafiltration device 12 is less than the diameter of the exosome in the introduced extraction solution;

The second-stage tangential flow ultrafiltration device 12 is connected to an inlet of the fast protein liquid chromatography system 2, and an outlet of the fast protein liquid chromatography system 2 is connected to the nanoparticle tracking analysis system 3;

The fast protein liquid chromatography system 2 is further connected to a freeze-drying refrigeration system, the freeze-drying refrigeration system comprises a mechanical transfer device 5, a freeze dryer 4, and a low-temperature storage space 6; and the mechanical transfer device 5 is used to move a product frozen by the freeze dryer 4 to the low-temperature storage space 6.

### Embodiment 2

The embodiment provides an exosome purification method, comprising the following steps:

S1. secondary tangential ultrafiltration: introducing an extraction solution containing the exosome into a first-stage tangential flow ultrafiltration device, recovering the ultrafiltered liquid through a first-stage ultrafiltration membrane to obtain from the permeate a first-stage liquid permeate, then enabling the first-stage liquid permeate to flow through the second-stage tangential flow ultrafiltration device continuously, retaining the exosome within the retentate as the pore size of the ultrafiltration membrane in the second-stage tangential flow ultrafiltration device is less than the diameter of the exosome, and recovering the retentate to obtain enriched liquid;

It is contemplated that the pore size of the ultrafiltration membrane in the first-stage tangential flow ultrafiltration device is greater than the pore size of the ultrafiltration membrane in the second-stage tangential flow ultrafiltration device, and the pore size of the ultrafiltration membrane in the second-stage tangential flow ultrafiltration device is less than the diameter of the exosome. More particularly, it is contemplated that the pore size of the ultrafiltration membrane in the first-stage tangential flow ultrafiltration device is 0.18 μm, and the pore size of the ultrafiltration membrane in the second-stage tangential flow ultrafiltration device is 0.03 μm.

S2. Enriched liquid pretreatment: conducting vacuum filtration or ultrasonic degassing treatment on the buffered solution mobile phase for 15 minutes to remove bubbles; firstly transferring an inlet pipe of the fast protein liquid chromatograph to a deionized water pump from 20% ethanol protection liquid for flushing, and then transferring to a buffered solution pump for flushing, with a flow rate of 0.7 mL/min and pump pressure alarm of 0.2 MPa;

Introducing the enriched liquid into the fast protein liquid chromatograph for chromatographic treatment to obtain eluent solution;

In the described embodiment, the fast protein liquid chromatograph is configured to chromatographically treat the enriched liquid solution using a chromatographic column employing an affinity column, the fast protein liquid chromatograph utilizing an equilibrium liquid comprising 0.06M Tris-HCl and 0.5 M NaCl having a PH of 7.9, an eluant comprising 0.06 M Tris-HCl, 0.5 M NaCl, and 0.6 M imidazole having a PH of 7.9, and a flow rate for equilibrating four column beds and eluting one column bed of 4 mL/min.

S3. Exosome particle size and concentration detection: flushing a detection window of the nanoparticle tracking analyzer with 1 mL of alcohol and 1 mL of pure water successively, then mixing the eluent solution obtained in the step S2 with a phosphate buffered solution in a volume ratio of 6:4, injecting the mixed solution into the nanoparticle tracking analyzer for analyzing and detecting the particle size and the concentration, and feeding back data results, detecting whether the concentration range of the exosome reaches 109/ml and the particle ranges from 30 nm to 150 nm; if the concentration range of the exosome reaches 109/ml and the particle ranges from 30 nm to 150 nm, entering step S4, or returning to the step S2 again for secondary tangential filtering treatment.

S4. Freeze-drying: conducting freeze-drying treatment on the enriched liquid or eluent solution to obtain freeze-dried powder, thus completing the purification of the exosome. The freeze-drying refrigeration system is composed of a freeze dryer, a mechanical arm, and a low-temperature storage. The enriched liquid or eluent solution is freeze-dried in the freeze dryer, then is sub-packaged into vials, and then is transferred to the low-temperature storage by the mechanical arm. The low-temperature storage is accompanied with a full alarm system.

### Embodiment 3

The embodiment provides an exosome purification method, comprising the following steps:

S1. secondary tangential ultrafiltration: introducing an extraction solution containing the exosome into a first-stage tangential flow ultrafiltration device, recovering the ultrafiltered liquid through a first-stage ultrafiltration membrane to obtain from the permeate a first-stage liquid permeate, then enabling the first-stage liquid permeate to flow through the second-stage tangential flow ultrafiltration device continuously, retaining the exosome within the retentate as the pore size of the ultrafiltration membrane in the second-stage tangential flow ultrafiltration device is less than the diameter of the exosome, and recovering the retentate to obtain enriched liquid;

The pore size of the ultrafiltration membrane in the first-stage tangential flow ultrafiltration device is greater than the pore size of the ultrafiltration membrane in the second-stage tangential flow ultrafiltration device, and the pore size of the ultrafiltration membrane in the second-stage tangential flow ultrafiltration device is less than the diameter of the exosome. It is expressly contemplated that the pore size of the ultrafiltration membrane in the first-stage tangential flow ultrafiltration device may be 0.19 μm, and the pore size of the ultrafiltration membrane in the second-stage tangential flow ultrafiltration device may be 0.02 μm.

S2. Enriched liquid pretreatment: conducting vacuum filtration or ultrasonic degassing treatment on the buffered solution mobile phase for 15 minutes to remove bubbles; firstly transferring an inlet pipe of the fast protein liquid chromatograph to a deionized water pump from 20% ethanol protection liquid for flushing, and then transferring to a buffered solution pump for flushing, with a flow rate of 0.7 mL/min, and pump pressure alarm of 0.2 MPa;

The enriched liquid in introduced into the fast protein liquid chromatograph for chromatographic treatment to obtain an eluent solution;

In the described embodiment, the fast protein liquid chromatograph is configured to chromatographically treat the enriched liquid solution using a chromatographic column employing an affinity column, the fast protein liquid chromatograph utilizing an equilibrium liquid comprising 0.06M Tris-HCl and 0.5 M NaCl having a PH of 7.9, an eluant comprising 0.06 M Tris-HCl, 0.5 M NaCl, and 0.6 M imidazole having a PH of 7.9, and a flow rate for equilibrating four column beds and eluting one column bed of 4 mL/min.

S3. Exosome particle size and concentration detection: flushing a detection window of the nanoparticle tracking analyzer with 1 mL of alcohol and 1 mL of pure water successively, then mixing the eluent solution obtained in the step S2 with a phosphate buffered solution in a volume ratio of 6:4, injecting the mixed solution into the nanoparticle tracking analyzer for particle size and concentration analysis and detection, and feeding back data results, detecting whether the concentration range of the exosome reaches 109/ml and the particle ranges from 30 nm to 150 nm; if the concentration range of the exosome reaches 109/ml and the particle ranges from 30 nm to 150 nm, entering step S4, or if not, returning to the step S2 again for secondary tangential filtering treatment;

S4. Freeze-drying: conducting freeze-drying treatment on the enriched liquid to obtain freeze-dried powder, thus completing the purification of the exosome. The freeze-drying refrigeration system is composed of a freeze dryer, a mechanical arm, and a low-temperature storage. The enriched liquid or eluent solution is freeze-dried in the freeze dryer, then is sub-packaged into vials, and then is transferred to the low-temperature storage by the mechanical arm. The low-temperature storage is accompanied with a full alarm system.

### Embodiment 4

The embodiment provides an exosome purification method, comprising the following steps:

S1. secondary tangential ultrafiltration: introducing an extraction solution containing the exosome into a first-stage tangential flow ultrafiltration device, recovering the ultrafiltered liquid through a first-stage ultrafiltration membrane to obtain from the permeate a first-stage liquid permeate, then enabling the first-stage liquid permeate to flow through the second-stage tangential flow ultrafiltration device continuously, retaining the exosome within the retentate as the pore size of the ultrafiltration membrane in the second-stage tangential flow ultrafiltration device is less than the diameter of the exosome, and recovering the retentate to obtain enriched liquid;

The pore size of the ultrafiltration membrane in the first-stage tangential flow ultrafiltration device is greater than the pore size of the ultrafiltration membrane in the second-stage tangential flow ultrafiltration device, and the pore size of the ultrafiltration membrane in the second-stage tangential flow ultrafiltration device is less than the diameter of the exosome. It is expressly contemplated that the pore size of the ultrafiltration membrane in the first-stage tangential flow ultrafiltration device may be 0.20 μm, and the pore size of the ultrafiltration membrane in the second-stage tangential flow ultrafiltration device may be 0.01 μm.

S2. Enriched liquid pretreatment: conducting vacuum filtration or ultrasonic degassing treatment on the buffered solution mobile phase for 15 minutes to remove bubbles; firstly transferring an inlet pipe of the fast protein liquid chromatograph to a deionized water pump from 20% ethanol protection liquid for flushing, and then transferring to a buffered solution pump for flushing, with a flow rate of 0.7 mL/min, and pump pressure alarm of 0.2 MPa;

The enriched liquid is introduced into the fast protein liquid chromatograph for chromatographic treatment to obtain an eluent solution;

In the described embodiment, the fast protein liquid chromatograph is configured to chromatographically treat the enriched liquid solution using a chromatographic column employing an affinity column, the fast protein liquid chromatograph utilizing an equilibrium liquid comprising 0.06M Tris-HCl and 0.5 M NaCl having a PH of 7.9, an eluant comprising 0.06 M Tris-HCl, 0.5 M NaCl, and 0.6 M imidazole having a PH of 7.9, and a flow rate for equilibrating four column beds and eluting one column bed of 4 mL/min S3.

Exosome particle size and concentration detection: flushing a detection window of the nanoparticle tracking analyzer with 1 mL of alcohol and 1 mL of pure water successively, then mixing the eluent solution obtained in the step S2 with a phosphate buffered solution in a volume ratio of 6:4, injecting the mixed solution into the nanoparticle tracking analyzer for particle size and concentration analysis and detection, and feeding back data results, detecting whether the concentration range of the exosome reaches 109/ml and the particle ranges from 30 nm to 150 nm; if the concentration range of the exosome reaches 109/ml and the particle ranges from 30 nm to 150 nm, entering step S4, or returning to the step S2 again for secondary tangential filtering treatment;

S4. Freeze-drying: conducting freeze-drying treatment on the enriched liquid to obtain freeze-dried powder, thus completing the purification of the exosome. The freeze-drying refrigeration system is composed of a freeze dryer, a mechanical arm, and a low-temperature storage. The enriched liquid or eluent solution is freeze-dried in the freeze dryer, then is sub-packaged into vials, and then is transferred to the low-temperature storage by the mechanical arm. The low-temperature storage is accompanied with a full alarm system.

Although the present disclosure is disclosed as above, the scope of protection of the present disclosure is not limited thereto. Various changes and modifications may be made by those skilled in the art without departing from the spirit and scope of the present disclosure, all of which should fall within the scope of protection of the present disclosure.

What is claimed is:

1. A method of exosome purification and characterization, comprising the steps of:
S1) introducing an extraction solution containing at least one exosome targeted for extraction into a first-stage tangential flow ultrafiltration device having a first-stage ultrafiltration membrane to obtain from the permeate a first-stage liquid permeate, delivering the first-stage liquid permeate into a second-stage tangential flow ultrafiltration device having a second-stage ultrafiltration membrane to obtain from the retentate an enriched liquid solution, wherein the pore size of the first-stage ultrafiltration membrane of the first-stage tangential flow ultrafiltration device is greater than the pore size of the second-stage ultrafiltration membrane of the second-stage tangential flow ultrafiltration device, and wherein the pore size of the second-stage ultrafiltration membrane of the second-stage tangential flow ultrafiltration device is less than the diameter of the at least one exosome targeted for extraction such thus the exosome targeted for extraction can pass through the first-stage ultrafiltration membrane and cannot pass through the second-stage ultrafiltration membrane;
S2) introducing the enriched liquid solution into a fast protein liquid chromatograph to obtain an eluent solution;
S3) mixing the eluent solution obtained in step S2 with a phosphate buffered solution to obtain a mixed solution, and injecting the mixed solution into a nanoparticle tracking analyzer to determine particle size and concentration data associated with the enriched liquid solution; and
S4) freeze drying the enriched liquid solution obtained in step S1 to obtain a freeze-dried powder permeate.

2. The exosome purification and characterization method according to claim 1, wherein in step S2, the pore size of the first-stage ultrafiltration membrane is greater than or equal to 0.18 μm, and the pore size of the second-stage ultrafiltration membrane is less than or equal to 0.03 μm.

3. The exosome purification and characterization method according to claim 1, wherein step S4 is performed via adding an excipient into the enriched liquid solution, bottling the enriched liquid solution containing the excipient, and then transferring the bottled enriched liquid solution containing the excipient into a vacuum freeze dryer to obtain the freeze-dried powder.

4. The exosome purification and characterization method according to claim 1, wherein in step S2, the fast protein liquid chromatograph is configured to chromatographically treat the enriched liquid solution using a chromatographic column employing an affinity column, the fast protein liquid chromatograph utilizing an equilibrium liquid comprising 0.06M Tris-HCl and 0.5 M NaCl having a PH of 7.9, an eluant comprising 0.06 M Tris-HCl, 0.5 M NaCl, and 0.6 M imidazole having a PH of 7.9, and a flow rate for equilibrating four column beds and eluting one column bed of 4 mL/min.

5. The exosome purification and characterization method according to claim 1, wherein in step S3, the volume ratio of the eluent solution to the phosphate buffered solution is 6:4.

6. The exosome purification and characterization method according to claim 1, wherein in step S1, the extraction solution containing at least one exosome targeted for extraction comprises one or more of: cell supernatant, blood, urine, saliva, amniotic fluid, urine, and seminal fluid.

7. The exosome purification and characterization method according to claim 1, wherein step S3 further comprises a step of comparing the determined particle size and concentration data to an acceptable particle size and concentration range, and if it is detecting that the determined particle size and concentration data is not within the acceptable particle size and concentration range, performing further tangential ultrafiltration treatment upon the enriched liquid solution, wherein the acceptable particle size and concentration range comprises a concentration greater than or equal to 109/ml, and a mean particle size range of between 30 nm to 150 nm.

8. An integrated device for exosome purification and characterization, the integrated device comprising:
a tangential flow ultrafiltration system comprising a first-stage tangential flow ultrafiltration device having a first-stage ultrafiltration membrane, and a second-stage tangential flow ultrafiltration device having a second-stage ultrafiltration membrane and a second-stage outlet, the first-stage tangential flow ultrafiltration device and the second-stage tangential flow ultrafiltration device being in fluid communication with one another, the first-stage ultrafiltration membrane having a greater pore size than the pore size of the second-stage ultrafiltration membrane,
a fast protein liquid chromatography system having a fast protein liquid chromatography inlet and a fast protein liquid chromatography outlet;
a nanoparticle tracking analysis system; and
a peristaltic pump for transferring liquid through the integrated device;
wherein the second-stage outlet is connected to the fast protein liquid chromatography inlet, and the fast protein liquid chromatography outlet is connected to the nanoparticle tracking analysis system.

9. The integrated device for exosome purification and characterization according to claim 8, wherein the first-stage tangential flow ultrafiltration device includes a first-stage inlet and a first-stage outlet, the second-stage tangential flow ultrafiltration device includes a second-stage inlet, and the first-stage outlet is in fluid communication with the second-stage inlet.

10. The integrated device for exosome purification and characterization according to claim 9, wherein the nanoparticle tracking analysis system is in fluid communication with the first-stage inlet.

11. The integrated device for exosome purification and characterization according to claim 8, wherein the fast protein liquid chromatography system is connected to a freeze-drying refrigeration system.

12. The integrated device for exosome purification and characterization according to claim 11, wherein the freeze-drying refrigeration system comprises a mechanical transfer device, a freeze dryer, and a low-temperature storage space, the mechanical transfer device being operative to transfer a product frozen by the freeze dryer to the low-temperature storage space.
